(19) United States
(12) Patent Application Publication
(10) Pub. No.: US 2026/0225982 A1
(43) Pub. Date: Aug. 6, 2026

(54) NEW METHOD FOR OZONOLYSIS AND REDUCTIVE QUENCHING OF OZONIDES

(71) Applicant: P2 SCIENCE, INC., Woodbridge, CT (US)

(72) Inventors: Patrick FOLEY, New Haven, CT (US); Yonghua YANG, East Lyme, CT (US); Prachiti BHATAWDEKAR, Hamden, CT (US)

(21) Appl. No.: 19/153,946

(22) PCT Filed: Feb. 6, 2024
(86) PCT No.: PCT/US2024/014662

§ 371 Date: Aug. 5, 2025

Related U.S. Application Data
(60) Provisional application No. 63/483,408, filed on Feb. 6, 2023.

(51) Int. Cl. C07C 45/40

(52) U.S. Cl. CPC C07C 45/40

(57) ABSTRACT

The present disclosure pertains to improved methods of performing ozonolysis by reductive quenching of the intermediate ozonides using glyoxal as the reductant.

### CROSS-REFERENCE TO RELATED APPLICATIONS

This application is an international application which claims priority to, and the benefit of, U.S. Provisional Application Ser. No. 63/483,408, filed on Feb. 6, 2023, the contents of which are hereby incorporated by reference in its entirety.

### FIELD OF INVENTION

The present disclosure pertains to improved methods of performing ozonolysis by reductive quenching of the intermediate ozonides using glyoxal as the reductant.

### BACKGROUND

Ozonolysis is an industrially useful transformation that involves the oxidation of an unsaturated carbon-carbon bond of an alkene using ozone. The reported mechanism (the “Criegee mechanism”) begins with initial formation of a primary ozonide (1,2,3-trioxolane) intermediate which rapidly decomposes into a carbonyl compound and carbonyl oxide compound. This pair of initial intermediates recombine to form a somewhat more stable secondary ozonide (1,2,4-trioxolane), a structure featuring a peroxide bridge.

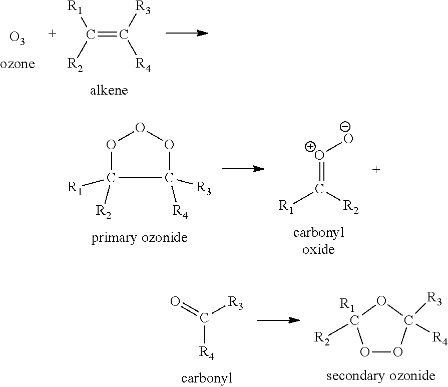

Although more stable than a primary ozonide or a carbonyl oxide, the secondary ozonide is still a high-energy chemical species subject to auto-accelerating thermal decomposition, decomposition to undesirable by-products, and organic peroxide formation (bis-peroxide, poly-peroxide, and hydroperoxide species). Therefore, further reactions must be carefully controlled in order to produce desired products in good yield.

Uncontrolled thermal decomposition of secondary ozonides typically yields highly variable mixtures of products due to the strong driving force of peroxide bond decomposition (highly exothermic) and unselective kinetic pathways such as radical propagation. For these reasons, the secondary ozonide is an undesirable chemical product and must be reacted in a subsequent chemical step.

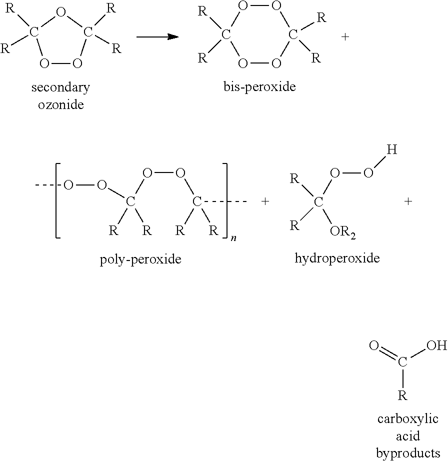

Traditionally, the secondary ozonide intermediates are either oxidatively or reductively cleaved to yield carbonyl products. Oxidative cleavage yields carboxylic acid and/or ketone products, while reductive cleavage yields ketone and/or aldehyde products. In addition, it is desirable to avoid the formation of peroxide products. For example, where R¹ and R³ in the above secondary ozonide are H, the following products may result:

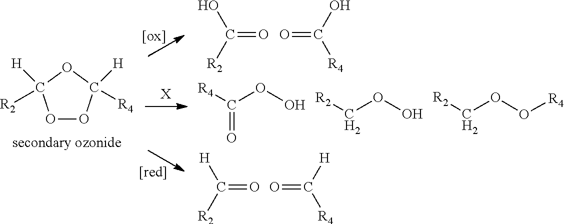

Commonly used reducing agents include sodium hydroxymethanesulfinate, hydroxymethanesulfinic acid, sulfite salts (e.g., sodium sulfite, sodium bisulfite, sodium metabisulfite, potassium sulfite, potassium bisulfite, potassium metabisulfite), sulfur dioxide, sodium dithionite, dimethyl sulfide, zinc, triphenylphosphine, thiourea, and formic acid, and hydrogenation methods over transition metal catalysts, such as palladium, platinum, and rhodium. However, each of these reagents has various drawbacks. For example, dimethyl sulfide has a noxious odor making it unpleasant to work with. Several of these reagents are toxic and/or environmentally unfriendly, which necessitates more costly procedures to ensure that waste streams are free of these agents. Some are very difficult to separate from the desired products (such as triphenylphosphine and its by-product triphenylphosphine oxide). Moreover, it is always beneficial from a cost effectiveness standpoint to develop less expensive reagents which are widely available for industrial use.

There is therefore a need for an improved method of reductive quenching of ozonides using an inexpensive, environmentally friendly reagent.

### BRIEF SUMMARY OF THE INVENTION

Glyoxal is a 2-carbon compound that is of intermediate oxidation state compared to its sister compounds ethylene glycol, glyoxylic acid, and oxalic acid:

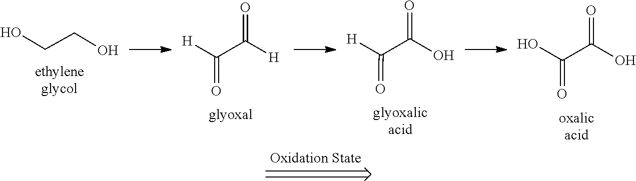

Glyoxal is commonly used as an organic chemistry building block, as a cross-linking agent in polymer chemistry, as a solubilizer, and as a fixative in histology. It has very low toxicity, with an oral rat LD50 of 3300 mg/kg (table salt has an LD50 of 3000 mg/kg). Glyoxylic acid is a versatile chemical reagent used in synthetic organic chemistry. Glyoxylic acid is also naturally occurring in many plant, bacterial, and protist cells as part of the glyoxylate cycle for generating carbohydrates from fatty acids. It too has low toxicity, with a rat LD50 of 2500 mg/kg.

Glyoxal and glyoxylic acid both predominantly exist in the form of their acetals or hemiacetals (e.g., hydrates) or as hemiacyl dimers or trimers. For example, in water, the following equilibria are established:

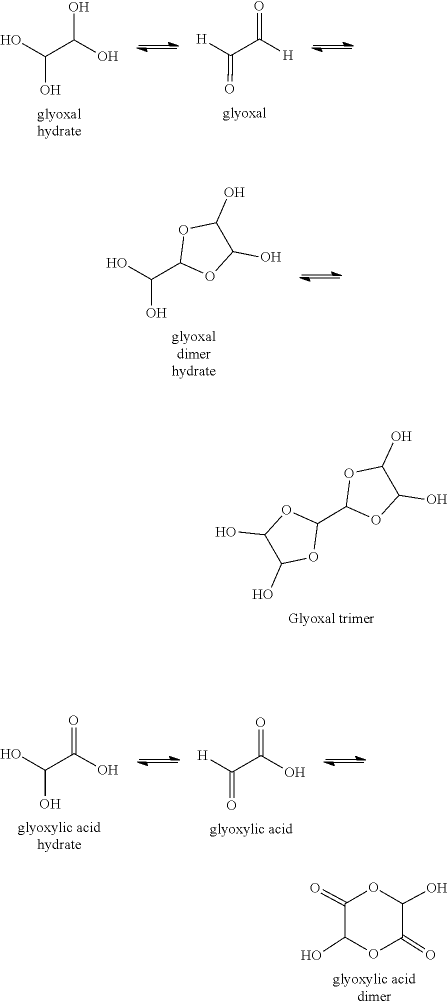

In an aqueous solution, the hydrates would predominate, while in the presence of an alcoholic solvent, the corresponding hemiacctals would predominate. Under strictly anhydrous conditions, the dimers and trimers would predominate.

Z. C. Sun et al., Ind. Eng. Chem. Res. 45:1849-52 (2006), disclose a synthesis of glyoxylic acid from glyoxal using hydroperoxide compounds as the oxidizing agent. The hydroperoxide compounds were generated in situ by the ozonolysis of maleic acid in the presence of reactive solvents, specifically, methanol, formic acid, or acetic acid. Because of its high nucleophilicity, the solvent traps the carbonyl oxide—the initially formed decomposition product of the primary ozonide intermediate—to form reactive hydroperoxides. The hydroperoxide then oxidizes the glyoxal to glyoxylic acid by an acid-catalyzed Bayer-Villager rearrangement mechanism, transferring the peroxy oxygen to the carbonyl of the glyoxal:

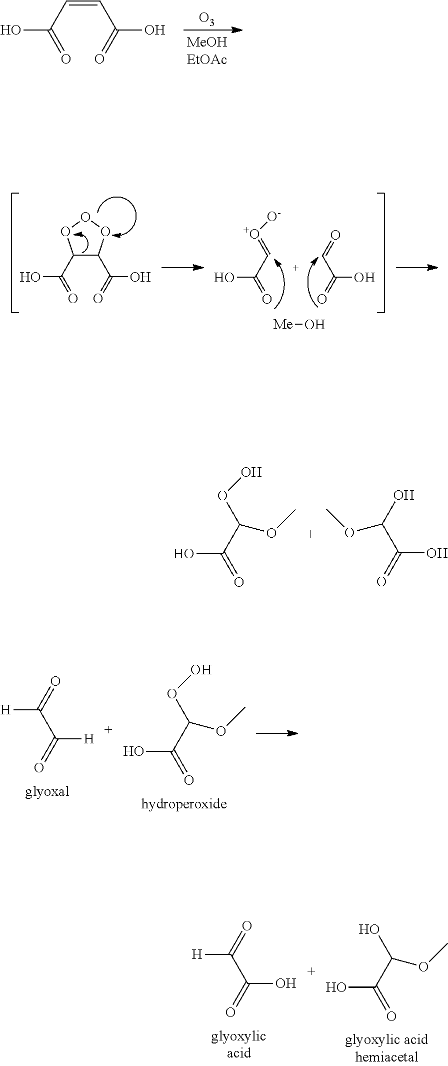

Sun discloses an example wherein 0.31 mol of maleic acid in 4:1 ethyl acetate:methanol is reacted 0.34 mol of ozone, and the resulting crude mixture (containing the hydroperoxide) was added to 0.30 mol of glyoxal to yield glyoxylic acid product. Under these conditions, Sun shows that there is 100% conversion of maleic acid to primary ozonide, and 99% conversion of the primary ozonide to hydroperoxide, and from the oxidizing effect of the hydroperoxide, there is 72% conversion and 80% yield of glyoxylic acid from glyoxal. Under the conditions described in Sun, using methanol as the reactive solvent, the primary ozonide is converted to a 1:1 mixture of 2-methoxy-2-hydroperoxyacetic acid and glyoxylic acid hemiacetal. The peroxyacetic acid compound is used as an oxidizing agent to form glyoxylic acid from glyoxal in good yield.

Sun does not, however, disclose the use of glyoxal as a reducing agent to decompose secondary ozonides, nor docs Sun teach or suggest that any other solvents would be suitable for forming the necessary hydroperoxide intermediates from the carbonyl oxide. Sun's teaching is limited to forming hydroperoxides using small, highly nucleophilic species such as methanol, formic acid and acetic acid.

Applicants have unexpectedly discovered that glyoxal is a versatile reducing agent for use in the reductive decomposition of primary and/or secondary ozonides, such as prepared from the ozonolysis of alkenes, e.g., unsaturated fatty acids, unsaturated fatty acid esters, and terpenes. Applicant's method is especially useful for the continuous quenching of ozonides produced by a flow ozonolysis reactor, such as a falling film reactor.

Thus, for example, the present disclosure provides methods as described herein, which are based on the following general scheme:

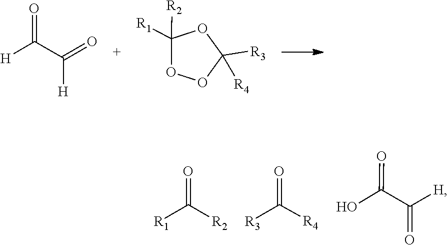

whereby glyoxal reacts, preferably, with a secondary ozonide, to yield ketone and/or aldehyde products and glyoxylic acid by-product. In further embodiments, the secondary ozonide above is optionally generated by the ozonolysis of an alkene according to the standard Criegee mechanism, as follows:

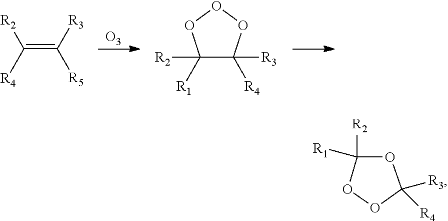

In the above alkene and ozonide structures, each of R₁, R₂, R₃ and R₄ is independently selected from H and C₁₋₂₅alkyl, or any two of R₁, R₂, R₃ and R₄ may combine to form a C₅₋₂₀ carbocyclic ring, wherein said alkyl and said ring are each independently optionally substituted by one or more groups selected from OH, C₁₋₆alkyl, C₂₋₁₂alkenyl, C₂₋₁₂alkynyl, C₃₋₁₂cycloalkyl, C₁₋₉-alkoxy, —O—R^{x}, —C(O)H, —C(O)—R^{x}, —C(O)—O—R^{x}, —O—C(O)—R^{x}, and wherein each R^{x} is independently selected from hydrogen, C₁₋₁₂alkyl, C₂₋₁₂alkenyl, C₂₋₁₂alkynyl, and C₃₋₁₂cycloalkyl.

In a first aspect, the present disclosure provides a method for reductive quenching of primary and/or secondary ozonides to yield aldehyde and/or ketone products using glyoxal as the reducing agent, wherein the method comprises the step of treating an ozonide mixture (e.g., primary and/or secondary ozonides) with glyoxal (e.g., 40% aqueous glyoxal).

In a second aspect, the present disclosure provides a method for performing ozonolysis with reductive quenching using glyoxal as the reducing agent, wherein the method comprises a first step of treating an alkene with ozone to form an ozonide mixture (e.g., primary and/or secondary ozonides), and a second step of treating the ozonide mixture from the first step with glyoxal (e.g., 40% aqueous glyoxal) to yield aldehyde and/or ketone products.

In another embodiment of the second aspect, the alkene is a monounsaturated or polyunsaturated fatty acid or fatty ester, or a monounsaturated or polyunsaturated terpene. In some embodiments, the alkene is a monounsaturated or polyunsaturated C₃₋₁₂ fatty acid or ester thereof. In some embodiments, the alkene is a fused bicyclic C₈₋₄₀ cycloalkene (e.g., C₈₋₄₀, C₈₋₃₀, C₈₋₂₀, C₁₀₋₄₀, C₁₀₋₃₀, C₁₀₋₂₀) with a bridgehead double bond, optionally substituted by one or more C₁₋₆alkyl groups.

In some embodiments of the first or second aspect, any aldehyde products may be isolated (e.g., by distillation) and then oxidized to carboxylic acid products.

### DETAILED DESCRIPTION OF THE INVENTION

In a first embodiment of the first aspect, the present disclosure provides a method (Method 1) for reductive quenching of primary and/or secondary ozonides to yield aldehyde and/or ketone products using glyoxal as the reducing agent, wherein the method comprises the step of treating an ozonide mixture (e.g., primary and/or secondary ozonides) with glyoxal (e.g., 40% aqueous glyoxal).

In further embodiments of the first aspect, the present disclosure provides:
1.1 Method 1, wherein the ozonide mixture comprising the primary and/or secondary ozonides is provided as a continuous stream from an ozonolysis operation (e.g., the output of the ozonolysis operation is the input for the reductive quenching), e.g., wherein the ozonolysis is carried out in a flow reactor, such as a co-current flow reactor, e.g., a falling film reactor, for example, as described in U.S. Pat. Nos. 10,071,944, 10,428,001, 10,934,239, or U.S. Pat. No. 10,668,446, the contents of each of which are hereby incorporated by reference in their entireties;
1.2 Method 1, or 1.1, wherein the primary and/or secondary ozonides are dissolved or suspended in a solvent mixture, e.g., a solvent mixture comprising one or more of water, a C₁₋₉ alkyl alcohol (e.g., tert-pentanol), C₁₋₁₂carboxylic acid (e.g., propanoic acid, nonanoic acid), a C₁₋₃alkyl C₁₋₁₂carboxylic ester (e.g., ethyl acetate, methyl hexanoate), or a C₁₋₆alkanediol (e.g., ethylene glycol), optionally an aqueous solvent mixture (e.g., water and an alcohol), optionally wherein the solvent mixture does not comprise methanol, formic acid, or acetic acid;
1.3 Method 1.2, wherein the solvent mixture comprises water and a C₁₋₉ alcohol;
1.4 Method 1.2, wherein the solvent mixture comprises water and a C₂₋₉ alcohol;
1.5 Method 1.2, wherein the solvent mixture comprises water and a C₃₋₉ alcohol;
1.6 Method 1.2, wherein the solvent mixture comprises water and a C₄₋₉ alcohol;
1.7 Method 1.2, wherein the solvent mixture comprises water and a C₅₋₆ alcohol;
1.8 Any of Methods 1.2-1.7, wherein the alcohol is selected from methanol, ethanol, propanol, isopropanol, n-butanol, isobutanol, s-butanol, t-butanol, n-pentanol, isopentanol (i.e., isoamyl alcohol), s-pentanol (i.e., 2-pentanol), t-pentanol (i.e., t-amyl alcohol), neopentyl alcohol, 2-methyl-1-butanol, 3-methyl-2-butanol, 3-pentanol, cyclopentanol, n-hexanol, 2-methyl-2-butanol, cyclohexanol, and 2-ethyl-2-hexanol;
1.9 Method 1.8, wherein the alcohol is selected from ethanol, propanol, isopropanol, n-butanol, isobutanol, s-butanol, t-butanol, n-pentanol, isopentanol (i.e., isoamyl alcohol), s-pentanol (i.e., 2-pentanol), t-pentanol (i.e., t-amyl alcohol), neopentyl alcohol, 2-methyl-1-butanol, 3-methyl-2-butanol, 3-pentanol, cyclopentanol, n-hexanol, 2-methyl-2-pentanol, and cyclohexanol;
1.10 Method 1.8, wherein the alcohol is a secondary or tertiary alcohol, e.g., selected from isopropanol, s-butanol, t-butanol, isopentanol, s-pentanol, 3-methyl-2-butanol, 3-pentanol, tert-pentanol, cyclopentanol, 2-methyl-2-pentanol, and cyclohexanol;
1.11 Method 1.10, wherein the alcohol is t-pentanol (i.e., t-amyl alcohol);
1.12 Method 1.2-1.11, wherein the solvent mixture is an aqueous solvent mixture comprising or consisting of a C₁₋₉ alkyl alcohol (e.g., tert-pentanol), C₁₋₁₂carboxylic acid (e.g., propanoic acid, nonanoic acid), a C₁₋₃alkyl C₁₋₁₂carboxylic ester (e.g., ethyl acetate, methyl hexanoate), or a C₁₋₆alkanediol (e.g., ethylene glycol), and water, wherein there is a 5:1 to 15:1 v/v ratio of said solvent (e.g., said alcohol, acid, ester, or diol) to water, e.g., a 6:1 to 12:1 ratio, or an 8:1 to 10:1 ratio, or about a 9:1 ratio, for example about a 9:1 ratio of t-amyl alcohol to water;
1.13 Method 1.2-1.12, wherein the ozonide mixture comprises or consists of unreacted alkene (e.g., the alkene that produced the ozonides by ozonolysis), primary and/or secondary ozonides, intermediates and by-products thereof, dissolved ozone and/or oxygen, and the solvent mixture (e.g., water and C₁₋₉ alcohol as defined in any preceding embodiment), optionally wherein the ozonide mixture does not comprise more than a trace amount of hydroperoxides;
1.14 Method 1, or any of 1.1-1.13, wherein the glyoxal is 40% aqueous glyoxal;
1.15 Method 1, or any of 1.1-1.14, wherein the ozonide mixture (e.g., primary and/or secondary ozonides), optionally in a solvent mixture (as defined in any preceding embodiment) is treated with a quenching solution, and the quenching solution comprises glyoxal and water (including any related species, such as glyoxal hydrate, glyoxal dimer, glyoxal dimer hydrate, and glyoxal trimer), e.g., 40% aqueous glyoxal;
1.16 Method 1.15, wherein the quenching solution does not comprise any other reducing agents;
1.17 Method 1.15, wherein the quenching solution does not comprise any oxidizing agents;
1.18 Method 1.15, wherein the quenching solution consists of glyoxal and water (including any related species, such as glyoxal hydrate, glyoxal dimer, glyoxal dimer hydrate, and glyoxal trimer), e.g., 40% aqueous glyoxal;
1.19 Method 1, or any of 1.1-1.18, wherein the glyoxal (e.g., the quenching solution) is added to the ozonide mixture;
1.20 Method 1, or any of 1.1-1.18, wherein the ozonide mixture is added to the glyoxal (e.g., the quenching solution);
1.21 Method 1, or any of 1.1-1.20, wherein the quenching is performed continuously, e.g., in a flow reactor, such as a flow reactor which continuously reacts in concurrent flow the ozonide mixture input and the quenching solution;
1.22 Method 1, or any of 1.1-1.20, wherein the ozonide mixture input is continuously added to an excess of quenching solution, e.g., in a continuously stirred tank reactor;
1.23 Method 1, or any of 1.1-1.22, wherein about 1.0-5.0 equivalents of glyoxal is used (e.g., is present in the quenching solution) based on the theoretical molar amount of ozonides present in the ozonide mixture, e.g., 1.5-5.0 equivalents, or 1.5-4.5 equivalents, or 1.5-4.0 equivalents, or 1.5-3.5 equivalents, or 1.5-3.0 equivalents, or 1.5-2.5 equivalents, or 2.0-5.0 equivalents, or 2.0-4.5 equivalents, or 2.0-4.0 equivalents, or 2.0-3.5 equivalents, or 2.0-3.0 equivalents, or 2.5-5.0 equivalents, or 2.5-4.5 equivalents, or 2.5-4.0 equivalents, or 2.5-3.5 equivalents, or 2.5-3.0 equivalents, about 2.7 equivalents;
1.24 Method 1, or any of 1.1-1.23, wherein the ozonide mixture is treated with the glyoxal (e.g., the quenching solution) at a temperature of 25° C. to 100° C., e.g., 30° C. to 90° C., or 40° C. to 80° C., or 50° C. to 80° C., or 60° C. to 80° C.; 1.25 Method 1, or any of 1.1-1.24, wherein upon completion of the quenching reaction (e.g., as determined by HPLC, GC, TLC, or peroxide testing), sodium chloride is added, and an aqueous extraction is performed, followed by distillation or crystallization to obtain the aldehyde and/or ketone product(s);
1.26 Method 1, or any of 1.1-1.25, wherein the ozonide mixture is derived from the ozonolysis of a monounsaturated or polyunsaturated terpene (e.g., monoterpenes, diterpenes, sesquiterpenes);
1.27 Method 1.26, wherein the terpene is selected from pinenes, camphenes, elemenes, citronellol, citronellal, citronellene, methoxycitronellene (7-methoxy-3,7-dimethyloct-1-ene), hydroxycitronellene (2,6-dimethyloct-7-en-2-ol), 2,3,7-trimethyloct-7-en-2-ol, isopulegol, longifolene, isothujone, thujone, valencene, myrcene, dihydromyrcene, dihydromyrcenol, limonene, carvone, linalool, geraniol, terpineol, squalene, and nootkatone;
1.28 Method 1, or any of 1.1-1.25, wherein the ozonide mixture is derived from the ozonolysis of a monounsaturated or polyunsaturated fatty acid or fatty acid ester;
1.29 Method 1, or any of 1.1-1.25, wherein the ozonide mixture is derived from the ozonolysis of a monounsaturated or polyunsaturated C₃₋₁₂ fatty acid or ester thereof;
1.30 Method 1, or any of 1.1-1.29, wherein the ozonide mixture is derived from the ozonolysis of a monounsaturated fatty acid, optionally cis or trans in configuration at the double bond;
1.31 Method 1, or any of 1.1-1.29, wherein the ozonide mixture is derived from the ozonolysis of a polyunsaturated fatty acid, optionally cis or trans in configuration at the double bonds;
1.32 Method 1, or any of 1.1-1.31, wherein the ozonide mixture is derived from the ozonolysis of a fatty acid selected from erucic acid, palmitoleic acid, oleic acid, elaidic acid, vaccenic acid, gondonic acid, paullinic acid, nervonic acid, stearidonic acid, gamma-linolenic acid, eicosapentaenoic acid, arachidonic acid, docosatetraenonic acid, linolenic acid, linoleic acid, and linoleaidic acid;
1.33 Method 1.32, wherein the fatty acid is selected from erucic acid and paullinic acid;
1.34 Method 1.32, wherein the fatty acid is selected from palmitoleic acid, oleic acid, elaidic acid, linolenic acid, and linoleic acid;
1.35 Method 1.32, wherein the fatty acid is erucic acid or oleic acid;
1.36 Method 1, or any of 1.1-1.31, wherein the ozonide mixture is derived from the ozonolysis of a fatty acid selected from myristoleic acid, sapienic acid, ricinoleic acid, and docosahexaenoic acid;
1.37 Any of methods 1.28-1.36, wherein the ozonide mixture is derived from the ozonolysis of an ester of said fatty acid;
1.38 Method 1.37, wherein the ester is a C₃₋₁₂ alkyl ester (e.g., isopropyl, sec-butyl, 2-ethylhexyl, or isodecyl ester) of the fatty acid, for example, methyl oleate, ethyl oleate, 2-ethylhexyl oleate, isodecyl oleate, methyl erucate, ethyl erucate, 2-ethylhexyl erucate, or isodecyl erucate;
1.39 Method 1, or any of 1.1-1.25, wherein the ozonide mixture is derived from the ozonolysis of an aliphatic or cyclic alkene (e.g., a monocyclic, bicyclic, or polycyclic cycloalkene), optionally monounsaturated, diunsaturated or polyunsaturated, and optionally substituted by one or more C₁₋₆alkyl groups;
1.40 Method 1.39, wherein the aliphatic or cyclic alkene is a C₅₋₂₀ alkene or a C₅₋₂₀ cycloalkene, e.g., a C₁₀₋₂₀ or a C₁₅₋₂₀ aliphatic or cyclic alkene, each optionally substituted by one or more C₁₋₆alkyl groups;
1.41 Method 1.39, wherein the aliphatic or cyclic alkene is selected from cyclopentene, cyclohexene, cycloheptene, cyclooctene, cyclononene, cyclodecene, cyclododecene, norbornene, and 2,3,4,5,6,7,8,9,10,11,12,13-dodecahydro-1H-cyclopenta[12]annulene, each optionally substituted by one or more C₁₋₆alkyl groups (e.g., 2-methyl-2,3,4,5,6,7,8,9,10,11,12,13-dodecahydro-1H-cyclopenta[12]annulene);
1.42 Method 1, or any of 1.1-1.25, wherein the ozonide mixture is derived from the ozonolysis of a fused bicyclic C₈₋₄₀ cycloalkene (e.g., C₈₋₄₀, C₈₋₃₀, C₈₋₂₀, C₁₀₋₄₀, C₁₀₋₃₀, C₁₀₋₂₀) with a bridgehead double bond, optionally substituted by one or more C₁₋₆alkyl groups, for example, a compound of the following formula:

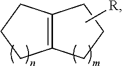

wherein there are 0-5 groups R, and each R is independently C₁₋₆alkyl, and n and m are integers independently selected from 1 to 17 (e.g., 1, 2, 3, 4, 5, 6, 7, 8, 9, or 10);
1.43 Method 1.42, wherein the fused bicyclic C₈₋₄₀ cycloalkene with a bridgehead 1.43 double bond is selected from 1,2,3,4,5,6-hexahydropentalene, 2,3,4,5,6,7-hexahydro-1H-indene, 1,2,3,4,5,6,7,8-octahydronaphthalene, 1,2,3,4,5,6,7,8-octahydroazulene, 2,3,4,5,6,7,8,9-octahydro-1H-cyclopenta[8]annulene, 1,2,3,4,5,6,7,8,9,10-decahydrobenzo[8]annulene, 2,3,4,5,6,7,8,9-octahydro-1H-benzo[7]annulene, 2,3,4,5,6,7,8,9,10,11-decahydro-1H-cyclopenta[10]annulene, and 2,3,4,5,6,7,8,9,10,11,12,13-dodecahydro-1H-cyclopenta[12]annulene, each optionally substituted with 0-5 groups R;
1.44 Method 1.42 or 1.43, wherein there are 0, 1, or 2 groups R;
1.45 Any of methods 1.39-1.44, wherein the one or more C₁₋₆alkyl groups are independently selected from methyl, ethyl, propyl, isopropyl, n-butyl, s-butyl, isobutyl, t-butyl, n-pentyl, s-pentyl, isopentyl, neopentyl, tert-pentyl, n-hexyl, s-hexyl, isohexyl, neohexyl, and tert-hexyl;
1.46 Any of methods 1.39-1.45, wherein the one or more C₁₋₆alkyl groups are independently selected from methyl, ethyl, propyl, and isopropyl;
1.47 Any of methods 1.39-1.46, wherein the one or more C₁₋₆alkyl groups are each methyl;
1.48 Any of methods 1.39-1.47, wherein the ozonide mixture is derived from the ozonolysis of 2-methyl-2,3,4,5,6,7,8,9,10,11,12,13-dodecahydro-1H-cyclopenta[12]annulene;
1.49 Method 1, or any of 1.1-1.48, wherein the ozonide mixture is not derived from the ozonolysis of an alpha, beta-unsaturated carboxylic acid (e.g., acrylic acid, maleic acid, fumaric acid, crotonic acid, isocrotonic acid, etc.);
1.50 Method 1, or any of 1.1-1.48, wherein the ozonide mixture is not derived from the ozonolysis of an ester of an alpha, beta-unsaturated carboxylic acid (e.g., acrylic acid, maleic acid, fumaric acid, crotonic acid, isocrotonic acid, etc.);
1.51 Method 1, or any of 1.1-1.48, wherein the ozonide mixture is not derived from the ozonolysis of maleic acid or an ester thereof;
1.52 Method 1, or any of 1.1-1.51, wherein the ozonide mixture is a homogenous solution, e.g., a homogenous aqueous solution comprising water and an alcohol (as described in any preceding embodiment) or a homogenous non-aqueous solution;
1.53 Method 1, or any of 1.1-1.51, wherein the ozonide mixture is an aqueous emulsion, e.g., an emulsion comprising water and an alcohol (as described in any preceding embodiment);
1.54 Method 1, or any of 1.1-1.51, wherein the ozonide mixture is neat (i.e., no added solvents);
1.55 Method 1, or any of 1.1-1.54, wherein the method provides only aldehyde products (e.g., no carboxylic acid or carboxylic ester products);
1.56 Method 1, or any of 1.1-1.54, wherein the method provides aldehyde and ketone products or only ketone products (e.g., no carboxylic acid or carboxylic ester products);
1.57 Method 1, or any of 1.1-1.56, wherein the method provides one or more of nonanal, 9-oxononanoic acid, a 9-oxononanoic acid ester (e.g., C₁₋₆ alkyl ester) brassyl aldehyde, a brassyl aldehyde ester (e.g., C₁₋₆ alkyl ester), 6-hydroxy-2,6-dimethylheptanal, 6-methoxy-2,6-dimethylheptanal, and 3-methylcyclopentadecane-1,5-dione;
1.58 Method 1, or any of 1.1-1.56, wherein the method provides a monocyclic diketone product, optionally substituted by one or more C₁₋₆alkyl groups, for example, a compound of the following formula:

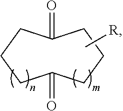

wherein there are 0-5 groups R, and each R is independently C₁₋₆alkyl, and n and m are integers independently selected from 1 to 17 (e.g., 1, 2, 3, 4, 5, 6, 7, 8, 9, or 10);
1.59 Method 1.58, wherein there are 0, 1, or 2 groups R;
1.60 Method 1.58 or 1.59, wherein the one or more C₁₋₆alkyl groups are independently selected from methyl, ethyl, propyl, isopropyl, n-butyl, s-butyl, isobutyl, t-butyl, n-pentyl, s-pentyl, isopentyl, neopentyl, tert-pentyl, n-hexyl, s-hexyl, isohexyl, neohexyl, and tert-hexyl;
1.61 Method 1.60, wherein the one or more C₁₋₆alkyl groups are independently selected from methyl, ethyl, propyl, and isopropyl;
1.62 Method 1.61, wherein the one or more C₁₋₆alkyl groups are each methyl;
1.63 Any of Methods 1.58-1.62, wherein the method provides cyclooctane-1,5-dione, cyclononane-1,5-dione, cyclodecene-1,6-dione, cyclodecane-1,5-dione, cycloundecane-1,5-dione, cycloundecane-1,6-dione, cyclododecane-1,6-dione, cyclotridecane-1,5-dione, or cyclopentadecane-1,5-dione, each optionally substituted with 0-5 groups R (e.g., methyl);
1.64 Method 1.63, wherein the method provides 3-methylcyclopentadecane-1,5-dione.

In a second aspect, the present disclosure provides a method (Method 2) for performing ozonolysis with reductive quenching using glyoxal as the reducing agent, wherein the method comprises a first step of treating an alkene with ozone to form an ozonide mixture (e.g., primary and/or secondary ozonides), and a second step of treating the ozonide mixture from the first step with glyoxal (e.g., 40% aqueous glyoxal) to yield aldehyde and/or ketone products. Optionally, the ozonolysis of the first step is conducted in a flow reactor: In further embodiments of the second aspect, the present disclosure provides:
2.1. Method 2, wherein the ozonide mixture comprising primary and/or secondary ozonides from the first step is provided as the input to the second step as a continuous stream;
2.2. Method 2, or 2.1, wherein the first step comprises the alkene dissolved or suspended in a solvent mixture, e.g., comprising one or more of water, a C₁₋₉ alkyl alcohol (e.g., tert-pentanol), C₁₋₁₂carboxylic acid (e.g., propanoic acid, nonanoic acid), a C₁₋₃alkyl C₁₋₁₂carboxylic ester (e.g., ethyl acetate, methyl hexanoate), or a C₁₋₆alkanediol (e.g., ethylene glycol), optionally an aqueous solvent mixture (e.g., water and an alcohol), optionally wherein the solvent mixture does not comprise methanol, formic acid, or acetic acid;
2.3. Method 2.2, wherein the solvent mixture comprises water and a C₁₋₉ alcohol;
2.4. Method 2.2, wherein the solvent mixture comprises water and a C₂₋₉ alcohol;
2.5. Method 2.2, wherein the solvent mixture comprises water and a C₃₋₉ alcohol;
2.6. Method 2.2, wherein the solvent mixture comprises water and a C₄₋₉ alcohol;
2.7. Method 2.2, wherein the solvent mixture comprises water and a C₅₋₆ alcohol;
2.8. Any of Methods 2.2-2.7, wherein the alcohol is selected from methanol, ethanol, propanol, isopropanol, n-butanol, isobutanol, s-butanol, t-butanol, n-pentanol, isopentanol (i.e., isoamyl alcohol), s-pentanol (i.e., 2-pentanol), t-pentanol (i.e., t-amyl alcohol), neopentyl alcohol, 2-methyl-1-butanol, 3-methyl-2-butanol, 3-pentanol, cyclopentanol, n-hexanol, 2-methyl-2-butanol, and cyclohexanol;
2.9. Method 2.8, wherein the alcohol is selected from ethanol, propanol, isopropanol, n-butanol, isobutanol, s-butanol, t-butanol, n-pentanol, isopentanol (i.e., isoamyl alcohol), s-pentanol (i.e., 2-pentanol), t-pentanol (i.e., t-amyl alcohol), neopentyl alcohol, 2-methyl-1-butanol, 3-methyl-2-butanol, 3-pentanol, cyclopentanol, n-hexanol, 2-methyl-2-pentanol, cyclohexanol, and 2-ethyl-2-hexanol;
2.10. Method 2.8, wherein the alcohol is a secondary or tertiary alcohol, e.g., selected from isopropanol, s-butanol, t-butanol, isopentanol, s-pentanol, 3-methyl-2-butanol, 3-pentanol, tert-pentanol, cyclopentanol, 2-methyl-2-pentanol, and cyclohexanol;
2.11. Method 2.8, wherein the alcohol is t-pentanol (i.e., t-amyl alcohol);
2.12. Method 2.2-2.11, wherein the solvent mixture is an aqueous solvent mixture comprising or consisting of comprising or consisting of a C₁₋₉ alkyl alcohol (e.g., tert-pentanol), C₁₋₁₂carboxylic acid (e.g., propanoic acid, nonanoic acid), a C₁₋₃alkyl C₁-12carboxylic ester (e.g., ethyl acetate, methyl hexanoate), or a C₁₋₆alkanediol (e.g., ethylene glycol), and water, wherein there is a 5:1 to 15:1 v/v ratio of said solvent (e.g., said alcohol, acid, ester, or diol) to water, e.g., a 6:1 to 12:1 ratio, or an 8:1 to 10:1 ratio, or about a 9:1 ratio, for example, about a 9:1 ratio of t-amyl alcohol to water.
2.13. Method 2.2-2.12, wherein the ozonide mixture from the first step comprises or consists of unreacted alkene (e.g., the alkene that produced the ozonides by ozonolysis), primary and/or secondary ozonides, intermediates and by-products thereof, dissolved ozone and/or oxygen, and the solvent mixture (e.g., water and C₁₋₉ alcohol as defined in any preceding embodiment), optionally wherein the ozonide mixture does not comprise more than a trace amount of hydroperoxides;
2.14. Method 2, or any of 2.1-2.13, wherein the glyoxal is 40% aqueous glyoxal;
2.15. Method 2, or any of 2.1-2.14, wherein the ozonide mixture (e.g., primary and/or secondary ozonides) from the first step, optionally in a solvent mixture (as defined in any preceding embodiment) is treated with a quenching solution, and the quenching solution comprises glyoxal and water (including any related species, such as glyoxal hydrate, glyoxal dimer, glyoxal dimer hydrate, and glyoxal trimer), e.g., 40% aqueous glyoxal;
2.16. Method 2.15, wherein the quenching solution does not comprise any other reducing agents;
2.17. Method 2.15, wherein the quenching solution does not comprise any oxidizing agents;
2.18. Method 2.15, wherein the quenching solution consists of glyoxal and water (including any related species, such as glyoxal hydrate, glyoxal dimer, glyoxal dimer hydrate, and glyoxal trimer), e.g., 40% aqueous glyoxal;
2.19. Method 2, or any of 2.1-2.18, wherein the glyoxal (e.g., the quenching solution) is added to the ozonide mixture;
2.20. Method 2, or any of 2.1-2.18, wherein the ozonide mixture from the first step is added to the glyoxal (e.g., the quenching solution);
2.21. Method 2, or any of 2.1-2.20, wherein the quenching is performed continuously, e.g., in a flow reactor, such as a flow reactor which continuously reacts in concurrent flow the ozonide mixture input from the first step and the quenching solution;
2.22. Method 2, or any of 2.1-2.20, wherein the ozonide mixture from the first step is continuously added to an excess of quenching solution, e.g., in a continuously stirred tank reactor;
2.23. Method 2, or any of 2.1-2.22, wherein about 1.0-5.0 equivalents of glyoxal is used (e.g., is present in the quenching solution) based on the molar amount of alkene reacted in the first step, e.g., 1.5-5.0 equivalents, or 1.5-4.5 equivalents, or 1.5-4.0 equivalents, or 1.5-3.5 equivalents, or 1.5-3.0 equivalents, or 1.5-2.5 equivalents, or 2.0-5.0 equivalents, or 2.0-4.5 equivalents, or 2.0-4.0 equivalents, or 2.0-3.5 equivalents, or 2.0-3.0 equivalents, or 2.5-5.0 equivalents, or 2.5-4.5 equivalents, or 2.5-4.0 equivalents, or 2.5-3.5 equivalents, or 2.5-3.0 equivalents, about 2.7 equivalents.
2.24. Method 2, or any of 2.1-2.23, wherein the ozonide mixture from the first step is treated with the glyoxal (e.g., the quenching solution) at a temperature of 25° C. to 100° C., e.g., 30° C. to 90° C., or 40° C. to 80° C., or 50° C. to 80° C., or 60° C. to 80° C. 2.25. Method 2, or any of 2.1-2.24, wherein upon completion of the quenching reaction (e.g., as determined by HPLC, GC, TLC, or peroxide testing), sodium chloride is added, and an aqueous extraction is performed, followed by distillation to obtain the aldehyde and/or ketone product(s);
2.26. Method 2, or any of 2.1-2.25, wherein the alkene is a monounsaturated or polyunsaturated terpene (e.g., monoterpenes, diterpenes, sesquiterpenes);
2.27. Method 2.26, wherein the terpene is selected from pinenes, camphenes, elemenes, citronellol, citronellal, citronellene, methoxycitronellene (7-methoxy-3,7-dimethyloct-1-ene), hydroxycitronellene (2,6-dimethyloct-7-en-2-ol), 2,3,7-trimethyloct-7-en-2-ol, isopulegol, longifolene, isothujone, thujone, valencene, myrcene, dihydromyrcene, dihydromyrcenol, limonene, carvone, linalool, geraniol, terpineol, squalene, and nootkatone;
2.28. Method 2, or any of 2.1-2.25, wherein the alkene is a monounsaturated or polyunsaturated fatty acid or fatty acid ester;
2.29. Method 2, or any of 2.1-2.25, wherein the alkene is a monounsaturated or polyunsaturated C₃₋₁₂ fatty acid or ester thereof;
2.30. Method 2, or any of 2.1-2.29, wherein the alkene is a monounsaturated fatty acid, optionally cis or trans in configuration at the double bond;
2.31. Method 2, or any of 2.1-2.29, wherein the alkene is a polyunsaturated fatty acid, optionally cis or trans in configuration at the double bonds;
2.32. Method 2, or any of 2.1-2.31, the alkene is a fatty acid selected from erucic acid, palmitoleic acid, oleic acid, elaidic acid, vaccenic acid, gondonic acid, paullinic acid, nervonic acid, stearidonic acid, gamma-linolenic acid, eicosapentaenoic acid, arachidonic acid, docosatetraenonic acid, linolenic acid, linoleic acid, and linoleaidic acid;
2.33. Method 2.32, wherein the fatty acid is selected from erucic acid and paullinic acid;
2.34. Method 2.32, wherein the fatty acid is selected from palmitoleic acid, oleic acid, elaidic acid, linolenic acid, and linoleic acid;
2.35. Method 2.32, wherein the fatty acid is erucic acid or oleic acid;
2.36. Method 2, or any of 2.1-2.31, wherein the alkene is a fatty acid selected from myristoleic acid, sapienic acid, ricinoleic acid, and docosahexaenoic acid;
2.37. Any of methods 2.28-2.36, wherein the alkene is an ester of said fatty acid;
2.38. Method 2.37, wherein the ester is a C₃₋₁₂ alkyl ester (e.g., isopropyl, sec-butyl, 2-ethylhexyl, or isodecyl ester) of the fatty acid, for example, methyl oleate, ethyl oleate, 2-ethylhexyl oleate, isodecyl oleate, methyl erucate, ethyl erucate, 2-ethylhexyl erucate, or isodecyl erucate;
2.39. Method 2, or any of 2.1-2.25, wherein the alkene is an aliphatic or cyclic alkene (e.g., a monocyclic, bicyclic, or polycyclic cycloalkene), optionally monounsaturated, diunsaturated or polyunsaturated, and optionally substituted by one or more C₁₋₆alkyl groups;
2.40. Method 2.39, wherein the aliphatic or cyclic alkene is a C₅₋₂₀ alkene or a C₅₋₂₀ cycloalkene, e.g., a C₁₀₋₂₀ or a C₁₅₋₂₀ aliphatic or cyclic alkene, each optionally substituted by one or more C₁₋₆alkyl groups;
2.41. Method 2.39, wherein the aliphatic or cyclic alkene is selected from cyclopentene, cyclohexene, cycloheptene, cyclooctene, cyclononane, cyclodecene, cyclododecene, norbornene, and 2,3,4,5,6,7,8,9,10,11,12,13-dodecahydro-1H-cyclopenta[12]annulene, each optionally substituted by one or more C₁₋₆alkyl groups (e.g., 2-methyl-2,3,4,5,6,7,8,9,10,11,12,13-dodecahydro-1H-cyclopenta[12]annulene);
2.42. Method 2, or any of 2.1-2.25, wherein the alkene is a fused bicyclic C₈₋₄₀ cycloalkene (e.g., C₈₋₄₀, C₈₋₃₀, C₈₋₂₀, C₁₀₋₄₀, C₁₀₋₃₀, C₁₀₋₂₀) with a bridgehead double bond, optionally substituted by one or more C₁₋₆alkyl groups, for example, a compound of the following formula:

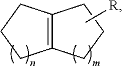

wherein there are 0-5 groups R, and each R is independently C₁₋₆alkyl, and n and m are integers independently selected from 1 to 17 (e.g., 1, 2, 3, 4, 5, 6, 7, 8, 9, or 10);
2.43. Method 2.42, wherein the fused bicyclic C₈₋₄₀ cycloalkene with a bridgehead double bond is selected from 1,2,3,4,5,6-hexahydropentalene, 2,3,4,5,6,7-hexahydro-1H-indene, 1,2,3,4,5,6,7,8-octahydronaphthalene, 1,2,3,4,5,6,7,8-octahydroazulene, 2,3,4,5,6,7,8,9-octahydro-1H-cyclopenta[8]annulene, 1,2,3,4,5,6,7,8,9,10-decahydrobenzo[8]annulene, 2,3,4,5,6,7,8,9-octahydro-1H-benzo[7]annulene,
2,3,4,5,6,7,8,9,10,11-decahydro-1H-cyclopenta[10]annulene, and
2,3,4,5,6,7,8,9,10,11,12,13-dodecahydro-1H-cyclopenta[12]annulene, each optionally substituted with 0-5 groups R;
2.44. Method 2.42 or 2.43, wherein there are 0, 1, or 2 groups R;
2.45. Any of methods 2.39-2.44, wherein the one or more C₁₋₆alkyl groups are independently selected from methyl, ethyl, propyl, isopropyl, n-butyl, s-butyl, isobutyl, t-butyl, n-pentyl, s-pentyl, isopentyl, neopentyl, tert-pentyl, n-hexyl, s-hexyl, isohexyl, neohexyl, and tert-hexyl;
2.46. Any of methods 2.39-2.45, wherein the one or more C₁₋₆alkyl groups are independently selected from methyl, ethyl, propyl, and isopropyl;
2.47. Any of methods 2.39-2.46, wherein the one or more C₁₋₆alkyl groups are each methyl;
2.48. Any of methods 2.39-2.47, wherein the alkene is 2-methyl-2,3,4,5,6,7,8,9,10,11,12,13-dodecahydro-1H-cyclopenta[12]annulene;
2.49. Method 2, or any of 2.1-2.48, wherein the alkene is not an alpha, beta-unsaturated carboxylic acid (e.g., acrylic acid, maleic acid, fumaric acid, crotonic acid, isocrotonic acid, etc.);
2.50. Method 2, or any of 2.1-2.48, wherein the alkene is not an ester of an alpha, beta-unsaturated carboxylic acid (e.g., acrylic acid, maleic acid, fumaric acid, crotonic acid, isocrotonic acid, etc.);
2.51. Method 2, or any of 2.1-2.48, wherein the alkene is not maleic acid or an ester thereof;
2.52. Method 2, or any of 2.1-2.51, wherein the ozonide mixture is a homogenous solution, e.g., a homogenous aqueous solution comprising water and an alcohol (as described in any preceding embodiment) or a homogenous non-aqueous solution;
2.53. Method 2, or any of 2.1-2.51, wherein the ozonide mixture is an aqueous emulsion, e.g., an emulsion comprising water and an alcohol (as described in any preceding embodiment);
2.54. Method 2, or any of 2.1-2.51, wherein the ozonide mixture is neat (i.e., no added solvents);
2.55. Method 2, or any of 2.1-2.54, wherein the first step comprises treating the alkene with gaseous ozone in a carrier gas in batch reactor;
2.56. Method 2, or any of 2.1-2.54, wherein the first step comprises treating the alkene with gaseous ozone in a carrier gas in a flow reactor, followed by (2) continuous quenching of the ozonolysis product stream by the second step;
2.57. Method 2.55 or 2.56, wherein the carrier gas is oxygen, nitrogen, or air.
2.58. Method 2.57, wherein the carrier gas is air;
2.59. Any of methods 2.56-2.58, wherein the ozone is present at a concentration of 2-5% in the carrier gas, e.g., 2-4%, or 3-5%, or 3-4%, such as 3-4% ozone in air;
2.60. Method 2, or any of 2.1-2.59, wherein the first step is carried out in a flow reactor, such as a co-current flow reactor, e.g., a falling film reactor, for example, as described in U.S. Pat. Nos. 10,071,944, 10,428,001, 10,934,239, or U.S. Pat. No. 10,668,446, the contents of each of which are hereby incorporated by reference in their entireties;
2.61. Method 2, or any of 2.1-2.59, wherein the first step occurs at a temperature from 0 to 25° C., e.g., 5 to 20° C., or 5 to 15° C., or about 10° C.;
2.62. Method 2, or any of 2.1-2.61, wherein the method provides only aldehyde products (e.g., no carboxylic acid or carboxylic ester products);
2.63. Method 2, or any of 2.1-2.61, wherein the method provides aldehyde and ketone products or only ketone products (e.g., no carboxylic acid or carboxylic ester products)
2.64. Method 2, or any of 2.1-2.63, wherein the method provides one or more of nonanal, 9-oxononanoic acid, a 9-oxononanoic acid ester (e.g., C₁₋₆ alkyl ester) brassyl aldehyde, a brassyl aldehyde ester (e.g., C₁₋₆ alkyl ester), 6-hydroxy-2,6-dimethylheptanal, 6-methoxy-2,6-dimethylheptanal, and 3-methylcyclopentadecane-1,5-dione;
2.65. Method 2, or any of 2.1-2.63, wherein the method provides a monocyclic diketone product, optionally substituted by one or more C₁₋₆alkyl groups, for example, a compound of the following formula:

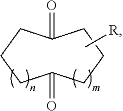

wherein there are 0-5 groups R, and each R is independently C₁₋₆alkyl, and n and m are integers independently selected from 1 to 17 (e.g., 1, 2, 3, 4, 5, 6, 7, 8, 9, or 10);
2.66. Method 2.65, wherein there are 0, 1, or 2 groups R;
2.67. Method 2.65 or 2.66, wherein the one or more C₁₋₆alkyl groups are independently selected from methyl, ethyl, propyl, isopropyl, n-butyl, s-butyl, isobutyl, t-butyl, n-pentyl, s-pentyl, isopentyl, neopentyl, tert-pentyl, n-hexyl, s-hexyl, isohexyl, neohexyl, and tert-hexyl;
2.68. Method 2.67, wherein the one or more C₁₋₆alkyl groups are independently selected from methyl, ethyl, propyl, and isopropyl;
2.69. Method 2.68, wherein the one or more C₁₋₆alkyl groups are each methyl;
2.70. Any of Methods 2.65-2.69, wherein the method provides cyclooctane-1,5-dione, cyclononane-1,5-dione, cyclodecene-1,6-dione, cyclodecane-1,5-dione, cycloundecane-1,5-dione, cycloundecane-1,6-dione, cyclododecane-1,6-dione, cyclotridecane-1,5-dione, or cyclopentadecane-1,5-dione, each optionally substituted with 0-5 groups R (e.g., methyl);
2.71. Method 2.70, wherein the method provides 3-methylcyclopentadecane-1,5-dione.

In a third aspect, the present disclosure provides for the use of glyoxal as a reducing agent for the reductive quenching of primary and/or secondary ozonides to yield aldehyde and/or ketone products, for example, a method according to Method 1 or any of 1.1-1.64.

In a fourth aspect, the present disclosure provides for the use of glyoxal as the reducing agent for performing ozonolysis with reductive quenching to yield aldehyde and/or ketone products, for example, a method according to Method 2 or any of 2.1-2.71.

In a fifth aspect, the present disclosure provides an aldehyde and/or ketone made according to Method 1 or any of 1.1-1.64, or according to Method 2 or any of 2.1-2.71.

In a sixth aspect, the present disclosure provides a product or composition comprising an aldehyde and/or ketone made according to Method 1 or any of 1.1-1.64, or made according to Method 2 or any of 2.1-2.71.

In some embodiments, the present Methods 1 and 2 relate to the quenching of the ozonide mixture resulting from ozonolysis of a fused bicyclic C₈₋₄₀ cycloalkene (e.g., C₈₋₄₀, C₈₋₃₀, C₈₋₂₀, C₁₀₋₄₀, C₁₀₋₃₀, C₁₀₋₂₀) with a bridgehead double bond, optionally substituted by one or more C₁₋₆alkyl groups. In these embodiments, the products are monocyclic cycloalkanediones, which are particularly useful. For example (wherein R is a C₁₋₆alkyl group, such as methyl):

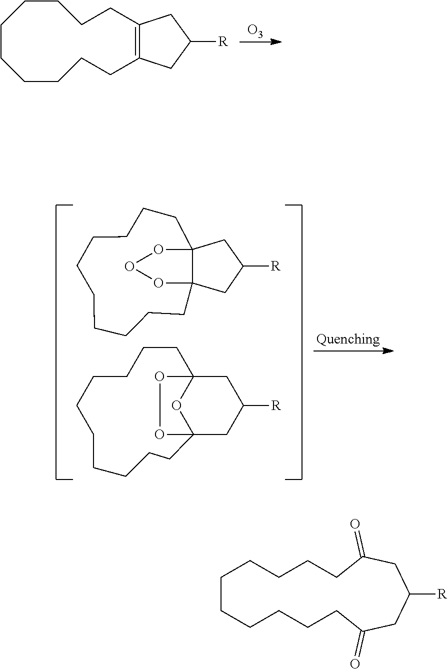

It is understood that in the term “bridgehead double bond,” the word “bridgehead” refers to the two carbon atoms which are common to any two adjacent fused rings, and this term is not intended to embrace the bridgehead carbon atom of any bridged bicyclic ring system.

In some embodiments, the present Methods 1 and 2 et seq. may be modified to provide that the initial products of the reductive quenching (the aldehyde and/or ketone products) may be separated (e.g., by distillation) to provide one or more aldehydes which is or are then subject to oxidation (e.g., separately) to form the corresponding carboxylic acid(s). This may be particularly useful, for example, where the separation of aldehyde/ketone products from each other would be easier than the separation of their corresponding carboxylic acid products from each, or where, for some other reason, direct oxidative quenching of an ozonolysis or ozonide product mixture is not desired. For example:

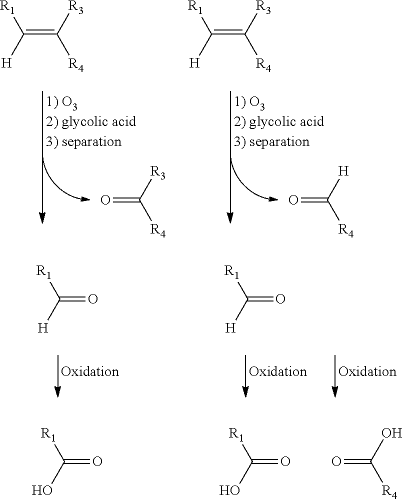

As an example, such a method could be applied as follows, in the ozonolysis of an erucic acid ester to provide a brassyl aldehyde ester and nonanal, followed by separation of the nonanal and oxidation of the brassyl aldehyde ester to a brassylic acid ester. In this particular example, the method is also shown with the initial step of converting erucic acid to the ester, the final step of hydrolyzing the brassylic acid ester to brassylic acid, and recycling of the alcohol used to from the ester:

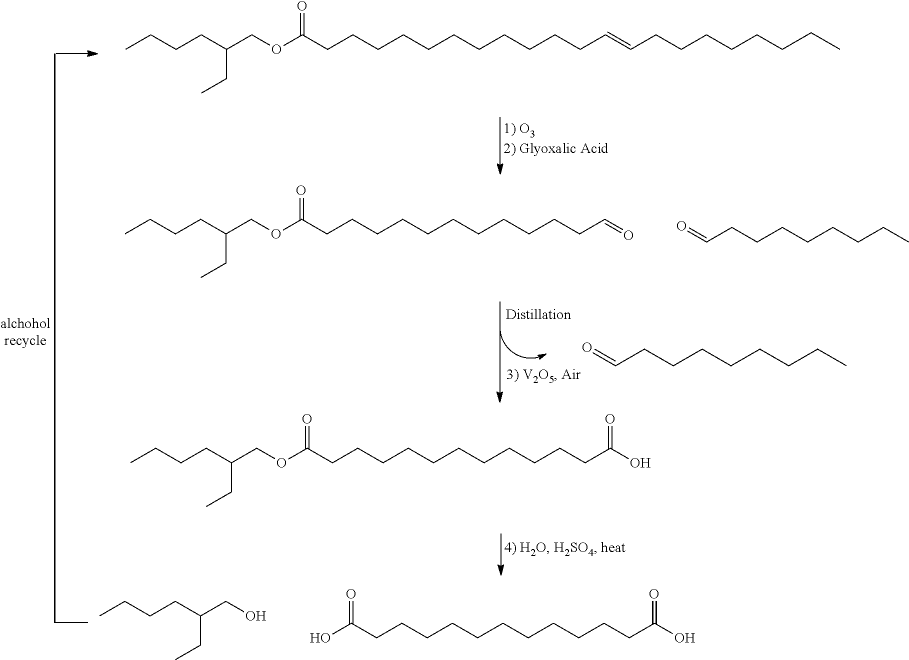

As used herein, the term “fatty acid” refers to a saturated or unsaturated, monocarboxylic acid, generally having from 4 to 28 carbon atoms. However, it is also understood that, unless indicated otherwise, the term “fatty acid” refers herein to a monounsaturated or polyunsaturated fatty acid having 7 to 28 carbon atoms.

The term “isodecyl” as used herein refers to any 10-carbon saturated alkyl chain that is not linear (i.e., not n-decyl). Examples of isodecyl groups include, but are not limited to, 2,4-dimethyloctan-2-yl, 2,6-dimethyl-octan-1-yl, 2,6-dimethyloctan-2-yl, 3,7-dimethyloctan-1-yl, and 3,7-dimethyloctan-3-yl.

It is understood that ozonolysis is a two-step process consisting of a first step which reacts an alkene with ozone to form a reactive ozonide intermediate and a second step which either oxidizes or reduces the ozonide mixture to provide carbonyl products. The terms “oxidative work-up” and “reductive work-up” refer to the conditions of this second step. It is understood that “oxidative work-up” exposes the reactive ozonides to an oxidizing reagent resulting in carboxylic acid/ketone products, while “reductive work-up” exposes the reactive ozonides to a reducing agent resulting in aldehyde/ketone products. It is further understood that either or both steps of the ozonolysis, unless provided otherwise herein, may be performed in a batch reactor or in flow reactor.

### EXAMPLES

### Example 1-2,6-dimethyl-6-hydroxyheptanal (hydroxymelonal)

A homogenous solution of dihydromyrcenol (750 g, 4.8 mol), t-amyl alcohol (2250 g, ~2800 mL) and water (300 g) is pumped through 2 sequential falling film reactors at a flowrate of 1.32 kg/h. The film is contacted with a co-current mixture of 3.7% w/w ozone in nitrogen gas with a total gas flow of 160 slpm (standard liters per minute). The tube reactor jacket temperature is maintained at a 10° C. setpoint. Continuous quenching of the ozonide stream from the reactor is performed by injecting the stream into a 40% aqueous solution of glyoxal in a continuous stirred tank reactor (CSTR) under nitrogen atmosphere. The total glyoxal loading is 2.66 molar equivalents to dihydromyrcenol. The temperature of the CSTR is set to 65° C., with the actual temperature staying in the range of 60-77° C.

After the quenching process is completed, the mixture is treated with sodium chloride solid (160 g, 2.75 mol) followed by phase separation. The top organic layer is then washed with NaOH solution (15%) and Sodium carbonate solution (10%) until pH of 8-9. Quantitative analysis by GC showed the yield was 78% after removing solvent after distillation.

### Example 2-2-Ethylhexyl Brassyl Aldehyde and Nonanal

2-Ethylhexyl erucate (450 g, 1 mol) is contacted with 5% ozone in air in a temperature-controlled reactor at 35° C. until ozone consumption ceased and the reaction exotherm abated. A 40% aqueous glyoxal solution (1.5 mol glyoxal) is then added to the reaction mixture slowly with stirring while maintaining the reaction temperature at 60° C. or below. Once the exotherm ceases, the reaction is stirred for an additional 2 hours at 60° C. to ensure completion of the reductive quenching. The reaction is checked by DSC and iodometric titration to confirm quenching. The phases are then separated and the organic phase is washed with water, then passed through a wiped film evaporator at reduced pressure to remove trace volatiles and to separate out the nonanal product, thus providing 2-ethylhexyl brassyl aldehyde in good yield and purity.

If desired, standard methods known to the artisan may be employed to convert the 2-ethylhexyl brassyl aldehyde to brassyl aldehyde. For example, a protection-hydrolysis-deprotection route, such as, forming an acetal, followed by base-catalyzed hydrolysis and deprotection of the acetal back to the aldehyde.

If desired, once the nonanal by-product has been removed by distillation, the 2-ethylhexyl brassyl aldehyde may be further oxidized, such as by using vanadium pentoxide and air, to afford 2-ethylhexyl brassylic acid monoester. Said monoester may further be hydrolyzed, such as using aqueous sulfuric acid, to afford brassylic acid in good yield.

Following the procedure of Example 1 or Example 2, the method of the present disclosure can be applied to the ozonolysis of methoxycitronellene to yield 6-methoxy-2,6-dimethylheptanal, oleic acid or an oleic acid ester to yield 9-oxononanoic acid or a 9-oxononanic acid ester, erucic acid or an erucic acid ester to yield brassyl aldehyde or a brassyl aldehyde ester, in good yield and purity.

### Example 3-3-Methylcyclopentadecane-1,5-dione

2-Methyl-2,3,4,5,6,7,8,9,10,11,12,13-dodecahydro-1H-cyclopenta[12]annulene (97 g) and propionic acid (194 g) are charged into a 500 mL jacketed, three-neck, round-bottom flask equipped with a thermometer, a gas purge sparger, and a condenser. The reaction is purged with ozone (5% ozone in air) until all of the raw material is consumed (monitored by GC), while the temperature of the reaction is controlled to between 12 and 25° C. The reaction is then purged with nitrogen for 2 minutes, and then the reaction mixture is transferred into a pressure equalizing dropping funnel connected with a 3-neck 1 L flask. This flask is also connected with another pressure equalizing dropping funnel and a condenser. The second dropping funnel is filled with aqueous glyoxal solution (81.6 g, 40%). The flask is pre-heated to 70° C. before adding both chemicals in parallel. The process is exothermic with the temperature maintained in a range of 70-90° C. during a period of 1 hour. When the addition is completed, and no further exotherm observed, the reaction mixture is cooled down. The mixture is treated with sodium chloride solution (20% w/v, 2×61 g) and then the solvents are removed by distillation under vacuum. The residue is dissolved in MTBE (200 mL) and washed with sodium carbonate solution. The organic solution is dried with anhydrous sodium sulfate and concentrated to yield 3-methylcyclopentadecane-1,5-dione (83.4 g, 75% mass yield; GC purity: 77%).

The Examples provided herein are exemplary only and are not intended to be limiting in any way to the various aspects and embodiments of the invention described herein.

1. A method for reductive quenching of primary and/or secondary ozonides to yield aldehyde and/or ketone products using glyoxal as the reducing agent, wherein the method comprises the step of treating an ozonide mixture (e.g., primary and/or secondary ozonides) with glyoxal (e.g., 40% aqueous glyoxal);
wherein the ozonide mixture is derived from the ozonolysis of a monounsaturated or polyunsaturated terpene, fatty acid, or fatty acid ester, or derived from the ozonolysis of an aliphatic or cyclic alkene.

2. The method according to claim 1, wherein the primary and/or secondary ozonides are dissolved or suspended in a solvent mixture, e.g., comprising one or more of water, a C₁₋₉ alkyl alcohol (e.g., tert-pentanol), C₁₋₁₂carboxylic acid (e.g., propanoic acid, nonanoic acid), a C₁₋₃alkyl C₁₋₁₂carboxylic ester (e.g., ethyl acetate, methyl hexanoate), or a C₁₋₆alkanediol (e.g., ethylene glycol), optionally an aqueous solvent mixture (e.g., water and an alcohol).

3. The method according to claim 2, wherein the solvent mixture does not comprise methanol, formic acid, or acetic acid.

4. The method according to claim 2, wherein the solvent mixture comprises water and an alcohol selected from isopropanol, s-butanol, t-butanol, isopentanol, s-pentanol, 3-methyl-2-butanol, 3-pentanol, tert-pentanol, cyclopentanol, 2-methyl-2-pentanol, and cyclohexanol.

5. The method according to claim 2, wherein the quenching is performed continuously on an ozonide mixture from a continuous ozonolysis operation.

6. A method for performing ozonolysis with reductive quenching using glyoxal as the reducing agent, wherein the method comprises a first step of treating an alkene with ozone to form an ozonide mixture (e.g., primary and/or secondary ozonides), and a second step of treating the ozonide mixture from the first step with glyoxal (e.g., 40% aqueous glyoxal) to yield aldehyde and/or ketone products;
wherein the alkene is a monounsaturated or polyunsaturated terpene, fatty acid, or fatty acid ester, or wherein the alkene is an aliphatic or cyclic alkene.

7. The method according to claim 6, wherein the first step comprises the alkene dissolved or suspended in a solvent mixture, e.g., comprising one or more of water, a C₁₋₉ alkyl alcohol (e.g., tert-pentanol), C₁₋₁₂carboxylic acid (e.g., propanoic acid, nonanoic acid), a C₁₋₃alkyl C₁₋₁₂carboxylic ester (e.g., ethyl acetate, methyl hexanoate), or a C₁-6alkanediol (e.g., ethylene glycol), optionally an aqueous solvent mixture (e.g., water and an alcohol).

8. The method according to claim 7, wherein the solvent mixture does not comprise methanol, formic acid, or acetic acid.

9. The method according to claim 7, wherein the solvent mixture comprises water and an alcohol selected from isopropanol, s-butanol, t-butanol, isopentanol, s-pentanol, 3-methyl-2-butanol, 3-pentanol, tert-pentanol, cyclopentanol, 2-methyl-2-pentanol, and cyclohexanol.

10. The method according to claim 6, wherein the alkene is a monounsaturated or polyunsaturated terpene, optionally wherein the terpene is selected from pinenes, camphenes, elemenes, citronellol, citronellal, isopulegol, longifolene, isothujone, thujone, valencene, myrcene, dihydromyrcene, limonene, carvone, linalool, geraniol, terpineol, squalene, and nootkatone.

11. The method according to claim 6, wherein the alkene is a monounsaturated or polyunsaturated fatty acid or fatty acid ester.

12. The method according to claim 11, wherein the alkene is a fatty acid selected from erucic acid, palmitoleic acid, oleic acid, elaidic acid, vaccenic acid, gondonic acid, paullinic acid, nervonic acid, stearidonic acid, gamma-linolenic acid, eicosapentaenoic acid, arachidonic acid, docosatetraenonic acid, linolenic acid, linoleic acid, linoleaidic acid, myristoleic acid, sapienic acid, ricinoleic acid, and docosahexaenoic acid, or a C₃₋₁₂ alkyl ester thereof.

13. The method according to claim 6, wherein the alkene is a monounsaturated or polyunsaturated terpene (e.g., monoterpenes, diterpenes, sesquiterpenes).

14. The method according to claim 13, wherein the terpene is selected from pinenes, camphenes, elemenes, citronellol, citronellal, citronellene, methoxycitronellene (7-methoxy-3,7-dimethyloct-1-ene), hydroxycitronellene (2,6-dimethyloct-7-en-2-ol), 2,3,7-trimethyloct-7-en-2-ol, isopulegol, longifolene, isothujone, thujone, valencene, myrcene, dihydromyrcene, dihydromyrcenol, limonene, carvone, linalool, geraniol, terpineol, squalene, and nootkatone.

15. The method according to claim 6, wherein the alkene is an aliphatic or cyclic alkene (e.g., a monocyclic, bicyclic, or polycyclic cycloalkene), optionally monounsaturated, diunsaturated or polyunsaturated, and optionally substituted by one or more C₁₋₆alkyl groups.

16. The method according to claim 15, wherein the alkene is selected from cyclopentene, cyclohexene, cycloheptene, cyclooctene, cyclononene, cyclodecene, cyclododecene, norbornene, and 2,3,4,5,6,7,8,9,10,11,12,13-dodecahydro-1H-cyclopenta[12]annulene, each optionally substituted by one or more C₁₋₆alkyl groups (e.g., 2-methyl-2,3,4,5,6,7,8,9,10,11,12,13-dodecahydro-1H-cyclopenta[12]annulene).

17. The method according to claim 6, wherein the alkene is a fused bicyclic C₈₋₄₀ cycloalkene (e.g., C₈₋₄₀, C₈₋₃₀, C₈₋₂₀, C₁₀₋₄₀, C₁₀₋₃₀, C₁₀₋₂₀) with a bridgehead double bond, optionally substituted by one or more C₁₋₆alkyl groups, for example, a compound of the following formula: 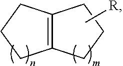
wherein there are 0-5 groups R, and each R is independently C₁₋₆alkyl, and n and m are integers independently selected from 1 to 17 (e.g., 1, 2, 3, 4, 5, 6, 7, 8, 9, or 10)

18. The method according to claim 1, wherein the method provides one or more of nonanal, 9-oxononanoic acid, a 9-oxononanoic acid ester (e.g., C₁₋₆ alkyl ester) brassyl aldehyde, a brassyl aldehyde ester (e.g., C₁₋₆ alkyl ester), 6-hydroxy-2,6-dimethylheptanal, 6-methoxy-2,6-dimethylheptanal, and 3-methylcyclopentadecane-1,5-dione.

19. The method according to claim 1, wherein the method provides cyclooctane-1,5-dione, cyclononane-1,5-dione, cyclodecene-1,6-dione, cyclodecane-1,5-dione, cycloundecane-1,5-dione, cycloundecane-1,6-dione, cyclododecane-1,6-dione, cyclotridecane-1,5-dione, or cyclopentadecane-1,5-dione, each optionally substituted with 0-5 groups R (e.g., methyl).

20. (canceled)

21. (canceled)

22. A product or composition comprising an aldehyde or ketone made according to the method of claim 1.

23. The method according to claim 1, wherein the monounsaturated or polyunsaturated terpene is selected from the group consisting of pinenes, camphenes, elemenes, citronellol, citronellal, citronellene, methoxycitronellene (7-methoxy-3,7-dimethyloct-1-ene), hydroxycitronellene (2,6-dimethyloct-7-en-2-ol), 2,3,7-trimethyloct-7-en-2-ol, isopulegol, longifolene, isothujone, thujone, valencene, myrcene, dihydromyrcene, dihydromyrcenol, limonene, carvone, linalool, geraniol, terpineol, squalene, and nootkatone, and wherein the monounsaturated or polyunsaturated fatty acid or ester is selected from the group consisting of erucic acid, palmitoleic acid, oleic acid, elaidic acid, vaccenic acid, gondonic acid, paullinic acid, nervonic acid, stearidonic acid, gamma-linolenic acid, eicosapentaenoic acid, arachidonic acid, docosatetraenonic acid, linolenic acid, linoleic acid, and linoleaidic acid, or any ester thereof, and wherein the aliphatic or cyclic alkene is selected from cyclopentene, cyclohexene, cycloheptene, cyclooctene, cyclononene, cyclodecene, cyclododecene, norbornene, and 2,3,4,5,6,7,8,9,10,11,12,13-dodecahydro-1H-cyclopenta[12]annulene, each optionally substituted by one or more C₁₋₆alkyl groups (e.g., 2-methyl-2,3,4,5,6,7,8,9,10,11,12,13-dodecahydro-1H-cyclopenta[12]annulene).
